(19) United States
(12) Patent Application Publication
(10) Pub. No.: US 2014/0315811 A1
(43) Pub. Date: Oct. 23, 2014

(54) OPHTHALMIC PREPARATIONS BASED ON PACAP (PITUITARY ADENYLATE CYCLASE ACTIVATING POLYPEPTIDE) WHICH RESTORE THE NORMAL VISUAL FUNCTION IN EARLY GLAUCOMA

(71) Applicants: Luciano Domenici, Budapest (HU); Marco Sanso', Budapest (HU); Luca Giovannini, Budapest (HU)

(72) Inventors: Luciano Domenici, Budapest (HU); Marco Sanso', Budapest (HU); Luca Giovannini, Budapest (HU)

(73) Assignee: HMFRA HUNGARY LIMITED LIABILITY COMPANY, Budapest (HU)

(21) Appl. No.: 14/009,594

(22) PCT Filed: Apr. 5, 2012
(86) PCT No.: PCT/EP2012/001514

§ 371 Date: Oct. 24, 2013

(30) Foreign Application Priority Data
Apr. 8, 2011 (IT) MI2011A000583

(51) Int. Cl. A61K 38/22

(52) U.S. Cl. CPC A61K 38/2278
USPC 514/13.1

(57) ABSTRACT

The present invention relates to ophthalmic preparations in the form of eyedrops based on PACAP (Pituitary Adenylate Cyclase Activating Polypeptide) which restore the normal visual function in retinal dystrophy/retinopathy and optic neuropathy, with special reference to glaucoma.

Said preparations can be administered topically to the intact eye surface, and are useful in the treatment of various forms of retinal dystrophy/retinopathy and optic neuropathy, such as glaucoma.

### TECHNICAL FIELD OF INVENTION

The present invention relates to ophthalmic preparations in the form of eyedrops comprising a peptide expressed in numerous areas of the central nervous system, including the retina, namely PACAP (Pituitary Adenylate Cyclase Activating Polypeptide).

Said preparations are useful to restore the normal visual function in retinal dystrophies/retinopathies and optic neuropathies such as glaucoma (congenital glaucoma, infantile glaucoma, juvenile glaucoma, adult glaucoma, chronic primary open-angle glaucoma, chronic primary angle-closure glaucoma, and secondary glaucoma including pigmentary glaucoma, iatrogenic glaucoma and acute glaucoma). In particular, the invention is designed to restore the normal visual function at the early stage of the glaucomatous disease, when the sequence of events leading to loss of the retinal ganglion cells is still reversible.

### PRIOR ART

PACAP, a peptide belonging to the VIP (vasoactive intestinal polypeptide)/glucagon/secretin family, is considered to be one of the most interesting peptides in view of its biological actions. The structure and functional properties thus conserved in the scale of vertebrates suggest that PACAP is involved in various vital functions; in fact, experimental findings have demonstrated its involvement in the development of various organs, the functions of the endocrine, cardiovascular, respiratory, reproductive and nervous systems, and the immune responses and circadian rhythms. It is synthesized in different areas of the CNS, including the retina, with a pleiotropic role, acting as neurotransmitter, neuromodulator or neurotrophic factor. PACAP is expressed in two forms: a longer 38-aminoacid peptide (PACAP 38) and a shorter 27-aminoacid peptide (PACAP 27), the longer form being the one mainly expressed in the nervous system. Two basic types of receptor are able to bind both PACAP 27 and 38: the class 1 receptor (PAC 1, with high affinity) and the class 2 receptor (with equal affinity for PACAP and VIP; two types of class 2 receptor can be distinguished: VPAC 1 and 2). After bonding to PAC1, PACAP induces the activation of an adenylate cyclase and phospholipase C, leading to an increase in cyclic AMP (cAMP) levels. PACAP can also be considered as a ligand of g-protein coupled receptors (GPCR), a family of receptors able to activate tyrosine kinase A and B (TrkA and TrkB respectively) in the absence of their specific ligands, NGF and BDNF respectively, leading to phosphorylation of multiple intracellular targets (Lee and Chao, 2002; Rajagopal et al., 2004).

The distribution of retinal cells able to synthesize and release PACAP appears to be limited to the ganglion cells, a sub-group of amacrine cells and horizontal cells (Seki et al. 2000; Hannibal and Fahrenkrug 2004); the distribution of the specific PAC1 receptor includes, in addition to ganglion cells, some types of amacrine cells and Müler cells (Seki et al. 1997; 2000; D'Agata and Cavallaro 1998; Kubrusly et al. 2005).

It has therefore been proposed that PACAP should be considered as an agent able to bond to two types of receptor present in the retina, PAC1 and PAC2, which exert a neuroprotective effect on the retinal cells that express them, especially the ganglion cells, and also the amacrine cells and Müler cells; in this specific case, “neuroprotective effect” refers to the ability of pharmacologically active molecules to oppose the cell death characteristic of numerous retinopathies. For example, PACAP (PACAP 38 and 27) has proved able to act as a neuroprotector, preventing damage to the retinal cells in various vascular disorders such as ischaemic retinopathy, and preventing the death of ganglion cells following a lesion of the optic nerve; PACAP also prevents the death of retinal cells caused by treatment with excitotoxins, such as glutamic acid and kainic acid, and cell death caused by irradiation with UV rays (Silveira et al., 2002; Seki et al. 2006; Atlasz et al. 2007; Atlasz et al, 2010).

These studies demonstrate that PACAP has been used in the field of neuroprotection, and more specifically to prevent the death of the retinal cells in various animal retinopathy models. Moreover, a direct action by PACAP on the affected retinal cells to restore their normal functionality, especially during an early neurodegenerative stage preceding cell death, has never been described.

It is known that in patients suffering from glaucoma and in experimental glaucoma models, the degeneration/death of the ganglion cells, with a consequent reduction in their retinal density, represents an advanced stage of glaucoma (Quigley et al. 1989; Buckingham et al. 2008; Boland and Quigley 2011). At an early stage of glaucoma, however, the ganglion cells are not yet so damaged as to be close to death, but begin to function abnormally, leading to a reduction in visual capacity; these functional alterations can be detected by recording the electroretinogram evoked by structured visual stimuli (pattern ERG, P-ERG), the source of which is in the inner nuclear layer, at the level of the ganglion cells (Maffei and Fiorentini, 1982; Maffei and Fiorentini, 1985). P-ERG has proved useful to detect early damage to the inner nuclear layer in both experimental animal models and patients suffering from intraocular hypertension or glaucoma (Domenici et al., 1991; Ventura and Porciatti 2006; Parisi et al. 2006; Falsini et al, 2008).

As regards the methods of administration of PACAP, recent data indicate that PACAP 38 and 27 are able to cross the blood-brain barrier, paving the way for their possible pharmacological application in systemic treatment (Nonaka et al., 2002; Dogrukol-Ak et al, 2009). However, systemic use of PACAP (by intravenous, intramuscular or intraperitoneal injection) should be avoided, because it can cause side effects, such as effects on the regulation of the circadian rhythms (Kawaguchi et al., 2010); in fact, PAC1 receptor knock-out mice present changes in the circadian rhythms (Hannibal et al. 2008). Moreover, systemic treatments with PACAP cannot completely prevent the degeneration of retinal cells in animal models, unlike intravitreal injections of PACAP, which release the molecule directly into the retina (Babai et al. 2006; Kiss et al. 2006). At present, therefore, there is a need to identify new PACAP preparations which can be administered by non-invasive techniques to convey PACAP to the retina, thus avoiding administration techniques such as intraocular, subretinal or retrobulbar injections, highly invasive methods which are unsuitable for chronic long-term treatment due to the associated risk of causing perforation of the eyeball, infection or haemorrhage, for example.

US 2008/0300182 describes ophthalmic preparations containing PACAP, PACAP 27 and 38, which are useful to prevent retinal cell death in various retinopathy models. The neuroprotective activity of PACAP has been demonstrated in vivo by intravitreal injection in an experimental model in which retinal cell death is pharmacologically induced; however, neither topical administration in the form of eyedrops nor the use of in vivo glaucoma models has been reported. Prevention of retinal cell death is attributed to the release of interleukin-6 (IL-6), an inflammatory cytokine, by the Müller retinal cells which express the PAC1 receptor. However, the PAC1 receptor is mainly concentrated in the inner nuclear layer, namely in the ganglion cells, as reported in the literature. Moreover, the increase in IL-6 in the retina, like other cytokines involved in the inflammatory processes, generates an alteration of the retinal response to light which can be measured with the flash electroretinogram (flash ERG; Ozawa et al., 2008); an increase in IL-6 may therefore induce, rather than treating, a further visual impairment.

Moreover, cell death in glaucoma represents a late stage of damage to the ganglion cells and optic nerve; by that time, the visual function is seriously impaired and difficult to restore.

U.S. Pat. No. 62,242,563 proposes PACAP analogues for the prevention of cell death in mammals and the treatment of various types of disorder (neurodegenerative diseases like Parkinson's disease and Alzheimer's disease, cardiovascular disease, diabetes, retinopathies and kidney disease). Prevention of cell death is stated to be responsible for the therapeutic activity in said disorders, among which retinopathies are only mentioned generically. The comments reported above for US 2008/0300182 therefore apply.

JP 10-505863 discloses the use of PACAP to prevent neuron death in various types of brain disease. Here again the objective is to prevent neuron death, so the comments made about US 2008/0300182 apply.

EP 1752158 discloses the use of PACAP or analogues thereof to promote the genesis of corneal cells to increase corneal sensitivity and treat patients suffering from “dry eye” and traumatic lesions of the corneal epithelium.

WO 200823717 discloses ophthalmic preparations containing PACAP to promote the secretion of tears by acting on the PAC1 receptor, and therefore does not relate to a retinopathy.

EP 1546198 discloses the use of PACAP to increase the proliferative and differentiating capacity of the stem cells of the basal prosencephalon so that said cells can be used to treat neurodegenerative disorders.

EP 1507551 discloses the use of PACAP to boost the proliferative/differentiating capacity of the neuronal progenitors present in the adult brain for the treatment of various alterations of the nervous system.

US 20020182729 discloses a method based on the use of PACAP to regulate the cycle cell in developing neurons and in the multiplication stage to treat various disorders of the nervous system.

Although effects of PACAP in the reduction and/or prevention of cell death have been suggested in some retinopathies and/or neurodegenerative disorders affecting other areas of the CNS, the therapeutic use of PACAP or its analogues to restore normal vision to glaucoma patients has not been described, nor can it be deduced from the state of the art.

Glaucoma is one of a series of progressive disorders affecting the eye which, if not suitably treated, lead to blindness due to loss of the ganglion cells and progressive atrophy of the optic nerve fibers. Glaucoma, especially its most frequent form, called primary open-angle glaucoma (POAG; 70-80% of cases of glaucoma), is characterised in most patients by increased intraocular pressure (IOP), with gradual narrowing of the aqueous humor drainage channels; if this chronic disorder is not rapidly diagnosed and treated it can lead, at an advanced stage, to ganglion cell death and damage to the optic nerve, and these alterations are practically irreversible. At the progressive stage, as well as the retina, glaucoma can affect the visual centers, such as the lateral geniculate body, eventually involving the visual cortex, by which stage treatment is useless. In recent years, pharmacological treatment has aimed at reducing the IOP, although a considerable number of patients are resistant to the current pharmacological treatment and suffer progressive, irreversible loss of visual function.

The pharmacological treatment of glaucoma, as reported above, necessarily requires a method of topical ocular administration that avoids side effects (systemic administrations) and the risk of eyeball perforation, infections or haemorrhage (intravitreal and subretinal administrations).

The retina is a partly separate part of the central nervous system; various types of barrier exist, including the blood-retinal barrier, which prevents the non-specific diffusion of compounds such as large molecules to the retina. The intraocular penetration of pharmacologically active compounds applied topically is regulated by barriers located in the cornea and the conjunctiva, the sclera, choroid and choroidal blood vessels, and by systemic absorption and metabolic breakdown effected by the enzymes present in those tissues. Once instilled, the pharmacologically active compounds must cross a complex system of barriers, including the blood-retinal barrier, to penetrate the underlying tissues as far as the retina.

Moreover, the retina, through the ganglion cells, from which the optic nerve fibers originate, is connected via the optic nerve to visual centers such as the dorsal part of the lateral geniculate body (dLGN).

It is therefore necessary to identify molecules, such as PACAP, which can be applied topically so as to avoid the risks associated with the use of invasive methods, and reach the retina in effective concentrations that not only inhibit the progress of the disorder induced by glaucoma in the ganglion cells and the optic nerve before their death, but above all restore the visual alterations to normal before they become irreversible.

### DESCRIPTION OF THE INVENTION

It has now been found that PACAP (or analogues thereof), when administered by topical ocular administration in glaucoma, restores the normal functionality of the retinal cells, especially the ganglion cells. PACAP therefore counteracts the visual impairment characteristic of an early stage of glaucoma, before ganglion cell death takes place.

The present invention is based on the demonstration that treatment with PACAP in a spontaneous murine model of glaucoma restores normal functionality in the inner nuclear layer (ganglion cells), leading to recovery of the visual capacity evaluated by P-ERG; this recovery occurs at a stage of the disease that precedes the stage characterised by ganglion cell death, which is evaluated by analyzing their retinal density.

The present invention therefore relates to ophthalmic formulations containing PACAP which are designed to restore the normal visual capacity in glaucoma.

The ophthalmic formulation according to the invention preferably takes the form of eyedrops containing PACAP (Pituitary Adenylate Cyclase Activating Polypeptide, PACAP 27 or PACAP 38).

The invention also relates to the use of PACAP (27 and 38) to prepare a medicinal product in the form of eyedrops for the treatment of neurodegenerative disorders of the retina, optic nerve and lateral geniculate body for the purpose of restoring normal vision at an early stage.

### LIST OF FIGURES

FIG. 1—Determination of cAMP levels in the retina following topical application of PACAP 38.

FIG. 2—Measurement of intraocular pressure (IOP) levels at various ages in an experimental model of spontaneous glaucoma (DBA/2J mouse).

FIG. 3—Density of ganglion cells in glaucoma, measured at different ages associated with increased IOP; study conducted in an experimental model of spontaneous glaucoma (DBA/2J mouse).

FIG. 4—Pattern electroretinogram (P-ERG) in the murine glaucoma model (DBA/2J mouse) at different ages.

FIG. 5—Topical application of PACAP 38 significantly reduces the alteration in the retinal response deriving from the ganglion cells in the inner nuclear layer during a glaucoma stage that precedes the reduction in density of the ganglion cells; study conducted in an experimental model of spontaneous glaucoma (DBA/2J mouse).

FIG. 6—Flash ERG recorded in the experimental model of spontaneous glaucoma (mouse DBA/2J) after topical treatment with PACAP to demonstrate the total absence of functional alterations in the photoreceptors and the outer nuclear layer.

### DETAILED DESCRIPTION OF THE INVENTION

The compositions according to the invention preferably take the form of solutions, suspensions, gels or ointments containing PACAP at concentrations of between 0.2 and 50 μg/μl (preferably 1-10 μg/μl). The therapeutic daily doses are approximately between 4 and 10000 μg (preferably 20-2000 μg), and may be reached in two or more daily administrations of a 10-100 μl ophthalmic solution instilled topically into the conjunctiva.

PACAP (27 and 38) can be administered alone or in combination with other active constituents, such as β-blockers, prostaglandins and carbonic anhydrase inhibitors.

The preparation will contain a suitable carrier which is pharmaceutically acceptable, compatible with the active ingredient, and tolerated by the eyes.

Examples of said carriers include saline solution, preferably containing 0.9% sodium chloride, and/or solutions containing viscosity-controlling agents such as carboxymethylcellulose, carbopol, hydroxypropylcellulose, polysaccharides, glycosaminoglycans and mixtures and derivatives thereof (such as salts).

The use of viscosity-controlling agents can improve bioavailability, guaranteeing a slower and more gradual passage of PACAP than when administered in saline solution, which is washed out of the conjunctiva more quickly.

The viscosity-controlling agent is preferably tamarind seed polysaccharide (TSP), a polysaccharide extracted from the seeds of the tamarind plant (Tamarindus indica) as disclosed in EP 0 892 636.

The TSP content can vary, preferably between 0.05 and 2% (weight/volume-w/v), and more preferably between 0.25 and 0.5% (w/v).

TSP is transparent in solution. The solutions are viscoelastic and sterile, and are used to protect the cornea and conjunctiva. TSP also forms a long-lasting film on the eye surface, which lubricates and moistens the cornea and conjunctiva.

According to another preferred aspect, the viscosified solution contains sodium carboxymethylcellulose in percentages ranging between 0.01 and 2% (w/v), preferably between 0.2 and 0.4% (w/v). Sodium carboxymethylcellulose is non-toxic and inert, and has a stable pH in solution. Moreover, a concentration of approx. 1% has a refraction index similar to that of tears.

A further preferred aspect is that the viscosified solution contains hyaluronic acid, more preferably hyaluronic acid in combination with TSP.

The hyaluronic acid content can range between 0.05% and 0.8% (w/v), preferably between 0.2 and 0.4% (w/v).

According to a preferred embodiment, the preparation contains PACAP at the concentration of 2 mg/mL in saline solution containing 0.9% NaCl.

According to another preferred embodiment, the preparation contains PACAP at a concentration of 2 mg/mL in saline solution containing 0.2% sodium carboxymethylcellulose.

According to a further preferred embodiment, the preparation contains PACAP at a concentration of 1 mg/mL in saline solution with 0.25% TSP.

The eyedrop preparation can be administered topically directly to the intact eye surface, i.e. by a non-invasive technique, avoiding the use of invasive methods such as intraocular, subretinal and retrobulbar injections. In particular, the preparation can be administered into the conjunctival sac. The preparation can also be formulated as an eyepatch or in contact lenses.

As demonstrated in the experimental section, when administered topically according to the invention, PACAP is conveyed to the retina, inducing an increase in the retinal concentration of the second cAMP messenger in the retinal cells, one of the intracellular targets of activation of the PACAP receptors, as reported in the introduction to the prior art. These results indicate that PACAP is not only able to pass through the various tissue barriers after topical treatment and spread to the retina, but also to retain a structure that enables it to bond to and activate its specific receptors expressed in various retinal cells, including the ganglion cells.

The formulations according to the invention can be used for the prevention and/or treatment of neurodegenerative disorders of the retina, optic nerve and lateral geniculate body, in particular the various forms of glaucoma (congenital glaucoma, infantile glaucoma, juvenile glaucoma, adult glaucoma, chronic primary open-angle glaucoma, chronic primary angle-closure glaucoma, and secondary glaucoma including pigmentary glaucoma, iatrogenic glaucoma and acute glaucoma).

The examples given below further illustrate the invention.

### EXAMPLE 1

### Determination of cAMP Levels in the Retina After 6 Hours' Topical Treatment of the Eye with PACAP-Based Preparations

The preparation Based on Saline Solution (0.9% NaCl) Containing PACAP 38 was Used

The test was conducted on mice (C57BL-6J, Harlan, Italy); PACAP 38 was applied topically, instilled into the conjunctival sac of one eye, while the other eye, used as control, was treated with the saline solution (“placebo”) only.
Determination of cAMP Levels in the Retina

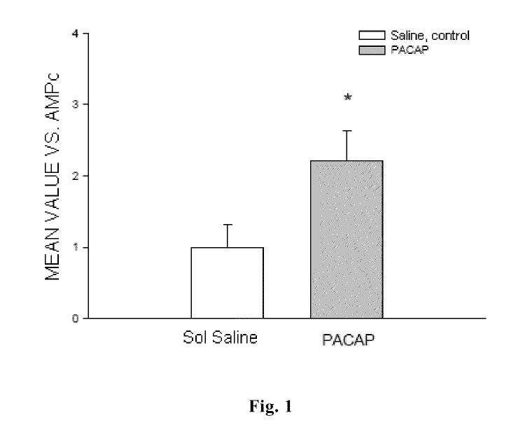

The animals were sacrificed 6 hours after the topical application, when deep anaesthesia had been induced by an intraperitoneal urethane injection (20%). The eye was then removed, and the cAMP level measured in the retinal homogenate of the eye treated with PACAP and of the other eye treated with saline only (control eye). The measurements were performed by cyclic AMP assay using the Cyclic AMP EIA KIT (Cayman Chemicon Company). The results shown in the chart in FIG. 1 were obtained with a topical application of PACAP (50 μM; 0.226 μg/μl) in saline solution (0.9% NaCl) and acetic acid (5%), and are expressed as relative values (%) compared with the control eye. The statistical analysis was conducted with Student's t-test, comparing the eye treated with PACAP with the control eye: in all cases the cAMP level was significantly higher in the treated eye than the control eye (*, p<0.05).

These results indicate that PACAP is able to cross the various barriers and reach the retina after topical application in the conjunctival sac; PACAP also seems able to activate its cell receptors which cause an increase in cAMP.

### EXAMPLE 2

### Effects Induced by Repeated Topical Application of PACAP at Various Ages in a Murine Glaucoma Model

The PACAP receptor, called PAC1, is known to be expressed in the ganglion cells (Seki et al. 1997; 2000; D'Agata and Cavallaro 1998; Hannibal and Fahrenkrug 2004; Kubrusly et al. 2005). The activity of PACAP was verified in the most common experimental model of spontaneous glaucoma, a double mutant mouse called DBA/2J (John et al., 1998; Chang et al., 1999). The DBA/2J mouse presents homozygous mutations of two separate genes; the first is tyrosine-related protein (Tyrpl−/−) coding for a melanosome protein, and the second is a membrane glycoprotein (Gpnmb−/−); pigment granules accumulate in the trabecular meshwork, similar to the form of pigmentary glaucoma, with consequent trabecular degeneration, atrophy of the iris and progressive impairment of the outflow of aqueous humor, which leads to an increase in IOP.

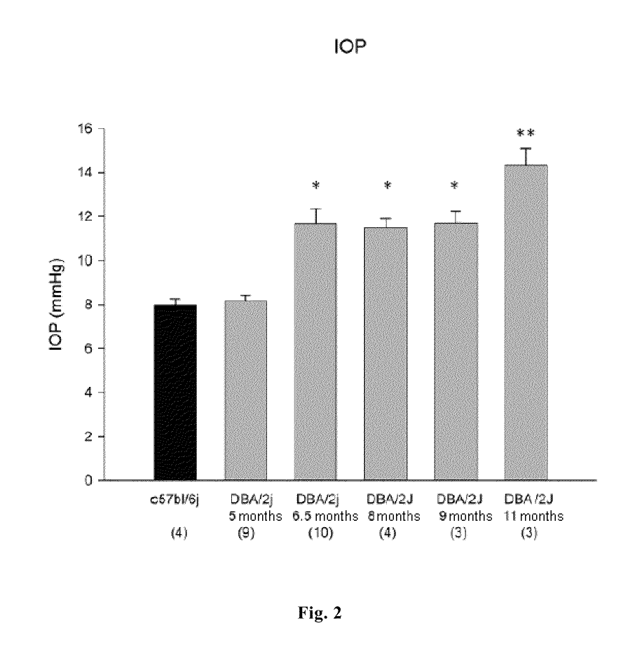

This mouse is characterised by a progressive increase in IOP, with progressive loss of the retinal response to structured visual stimuli (which may be lattices formed by pale strips alternating with dark strips, or chess pieces, with different contrasts and sizes) which depends on the inner nuclear layer/ganglion cells; in humans and in the animal model, this retinal response is called the pattern electroretinogram (P-ERG; Domenici et al.,1991; Ventura and Porciatti, 2006; Falsini et al., 2008). The dysfunction of the ganglion cells is followed at a late stage by degeneration of the ganglion cells, with a reduction in their density and progressive atrophy of the optic nerve (Ventura et al., 2006; Buckingham et al., 2008). As shown in FIG. 2, in this murine glaucoma model (DBA/2J), the IOP starts to increase between 5 and 6 months of postnatal life: at 6½ months the IOP in the DBA/2J mouse already appears significantly higher (t-test;* p<0.05) than that measured in the normal mouse(C57bl/6J) and in the DBA/2J mouse at the age of 5 months, i.e. before the onset of the disorder. At the age of 11 months the IOP rises further; a progression of the disorder similar to that which takes place in human patients can therefore be deduced.

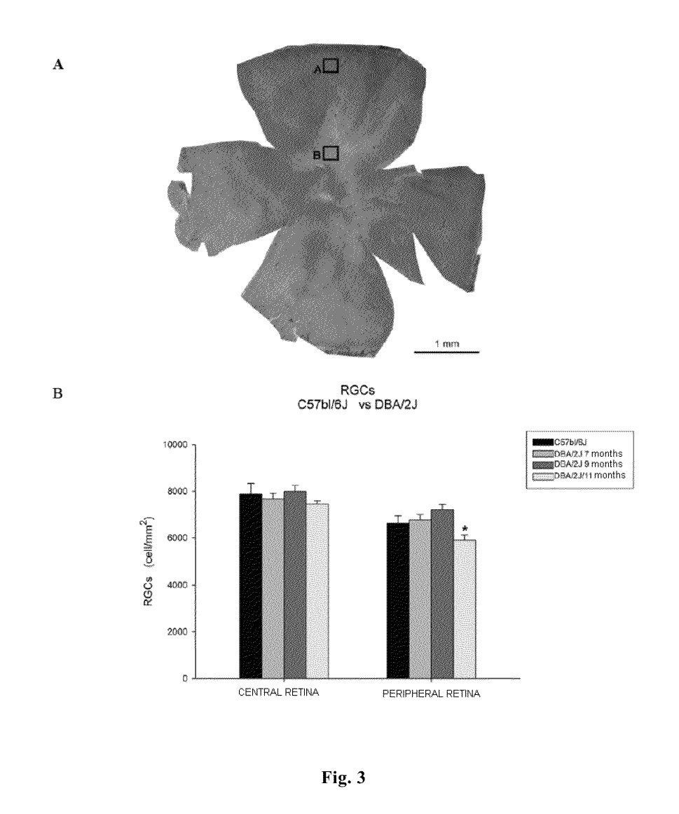

We evaluated whether an increase in IOP corresponds to an increase in cell death and a consequent loss of ganglion cells, whose density reduces. The graph in FIG. 3 shows the density of the ganglion cells measured by confocal microscope analysis of the distribution of the ganglion cells labelled with a fluorescent antibody able to detect ã-Synuclein (Surgucheva et al., 2008); the graph shows that a slight reduction in the density of the ganglion cells, starting from the peripheral retina, only begins to appear at the advanced age of 11 months. These results demonstrate that the death of the ganglion cells begins to be manifested at the advanced age of 11 months in the murine model of glaucoma, the DBA/2J mouse, namely approx. 5 months after the initial increase in IOP; Buckingham et al. (2008) suggest that the maximum level of cell death takes place after 16 months in the DBA/2J mouse. The studies previously conducted on patients suffering from glaucoma are in line with those reported in the murine model, suggesting that the visual impairment begins to be manifested at an early stage, before the density of the ganglion cells is reduced and the optic nerve fibers atrophy (Quigley, 1989; Harwerth, Quigley, 2006).

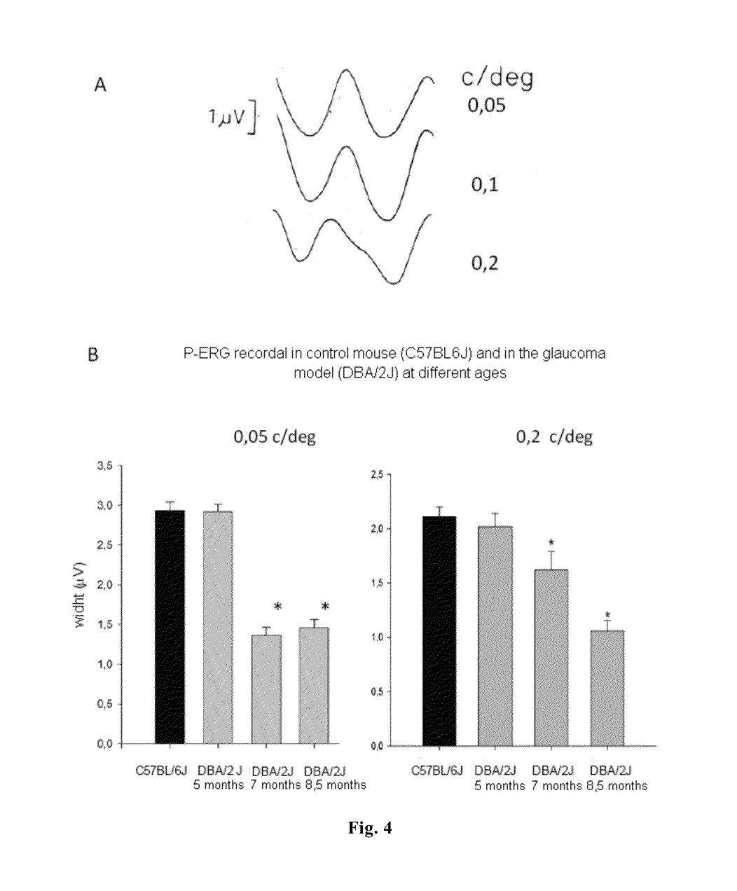

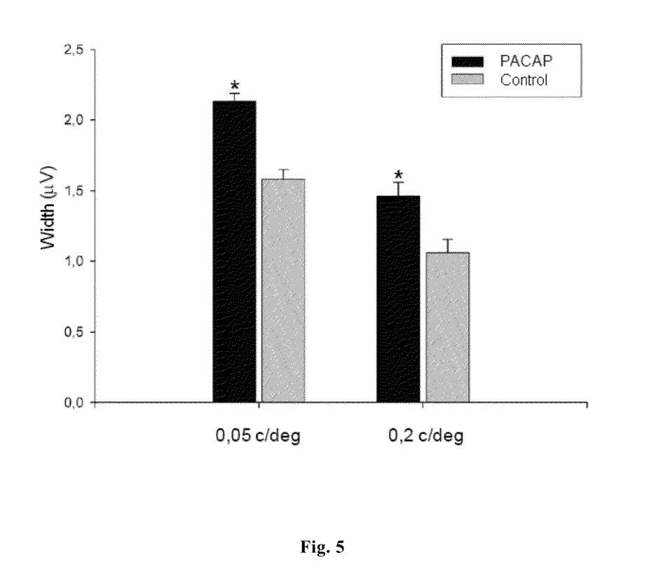

The early visual impairments caused by abnormal functioning of the retinal ganglion cells, especially their synapses, are easily measurable in patients and in animal models by recording the pattern electroretinogram (P-ERG), a non-invasive method able to detect functional alterations, even at the initial stage, of the inner nuclear layer, and in particular of the ganglion cells (Ventura et al. 2006; Falsini et al., 2008; 2009). FIG. 4 shows the P-ERG evoked by structured visual stimuli (the visual lattices used as stimulus were horizontal lattices, the luminance profile of which was characterised by spatial frequencies of 0.05, 0.1 and 0.2 cycles/degree, and 90% contrast which was inverted at the time frequency of 2 Hz) recorded by corneal electrodes wired to an amplifier and connected to a computer for online analysis. As shown in FIG. 4, the P-ERG is already altered in the 7-month-old DBA/2J mouse (a significant reduction in the P-ERG amplitudes evoked by visual stimuli with a spatial frequency of 0.05 and 0.2 c/deg; Student's t-test* p<0.05) in correspondence with the increased IOP (FIG. 2). At the age of 8 months, i.e. when the IOP had increased on a stable basis (FIG. 2) but there was no reduction in the density of the ganglion cells (FIG. 3), two weeks' treatment with repeated topical applications of PACAP in saline solution was given (one treatment every 48 hours) in one eye and the carrier (saline solution) in the other (control eye). Three different PACAP concentrations were used (N=3 DBA/2J mice per group): 500 nM (0.0022 μg/μl), 1 μM (0.0045 μg/μl), 50 μM (0.226 μg/μl); only the highest concentration (50 μM, 0.226 μg/μl) proved effective. The histogram in FIG. 5 shows that topical treatment with PACAP at the concentration of 50 μM (0.226 μg/μl) for 2 weeks between 8 and 8.5 months (one application every 48 hours) prevented the alteration of P-ERG in the DBA/2J mouse, arresting the progress of the disorder (compare the data for the treated eye with the control eye; Student's t-test, *p<0.05) and restoring the visual response of the ganglion cells of the inner nuclear layer to normal.

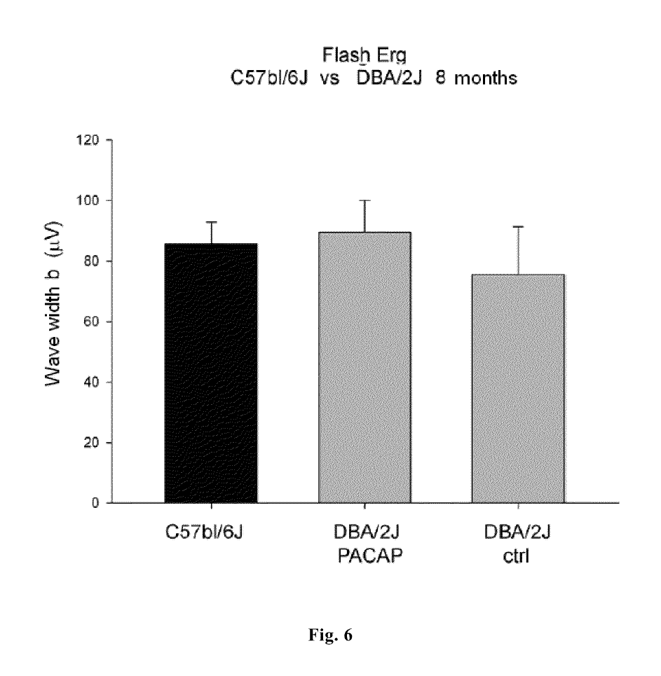

The other type of electroretinogram, flash ERG, which measures the response of the photoreceptors and the outer nuclear layer to light, appears wholly intact in glaucoma, further supporting the theory that this is a type of disorder affecting the inner nuclear layer, namely the ganglion cells, at least at the early stage. FIG. 6 shows the amplitudes of the response of the outer nuclear layer (photoreceptors and post-receptor cells) to light (flash ERG), which indicates that in the untreated DBA/2J mouse the response of the outer nuclear layer to light is present and perfectly normal. A second result, shown in FIG. 6, is particularly important: the eye of the DBA/2J mouse, treated topically with PACAP (50 μM) in saline solution (two weeks, one treatment every 48 h), presents a normal flash ERG. This second result suggests that PACAP is unable to modify the photopigment contained in the outer segment of the photoreceptors, and consequently to alter the response of the photoreceptors to light (flash ERG), as would be expected if this type of treatment induced an increase in IL-6 at retinal level (Ozawa et al. 2008), as proposed in US 2008/0300182.

The data reported lead to the conclusion that repeated topical treatment with PACAP at effective concentrations prevents functional alterations of the ganglion cells and restores the retinal visual capacity in an experimental glaucoma model at an early stage. The minimal effective concentration of PACAP able to exert protective effects on the ganglion cell function was 0.226 μg/μl.

### REFERENCES

Atlasz T, Babai N, Kiss P, Reglodi D, Tamás A, Szabadfi K, Tóth G, Hegyi O, Lubics A, Gábriel R. Pituitary adenylate cyclase activating polypeptide is protective in bilateral carotid occlusion-induced retinal lesion in rats. Gen Comp Endocrinol. 2007 August-September; 153(1-3):108-14.

Atlasz T, Szabadfi K, Kiss P, Tamas A, Toth G, Reglodi D, Gabriel R. Evaluation of the protective effects of PACAP with cell-specific markers in ischemia-induced retinal degeneration. Brain Res Bull. 2010 Mar. 16; 81 (4-5):497-504.

Babai N, Atlasz T, Tamás A, Reglödi D, Tóth G, Kiss P, Gábriel R. Degree of damage compensation by various PACAP treatments in monosodium glutamate-induced retinal degeneration. Neurotox Res. 2005 November; 8(3-4):227-33.

Boland M V, Quigley H A. Evaluation of a combined index of optic nerve structure and function for glaucoma diagnosis. BMC Ophthalmol. 2011 Feb. 11; 11(1):6.

Buckingham B P, Inman D M, Lambert W, Oglesby E, Calkins D J, Steele M R, Vetter M L, Marsh-Armstrong N, Horner P J. Progressive ganglion cell degeneration precedes neuronal loss in a mouse model of glaucoma. J Neurosci. 2008 Mar. 12; 28(11):2735-44.

Domenici L, Gravina A, Berardi N, Maffei L. Different effects of intracranial and intraorbital section of the optic nerve on the functional responses of rat retinal ganglion cells. Exp Brain Res. 1991; 86(3):579-84.

D'Agata V, Cavallaro S. Functional and molecular expression of PACAP/VIP receptors in the rat retina. Brain Res Mol Brain Res. 1998 February; 54(1):161-4.

Dogrukol-Ak D, Kumar V B, Ryerse J S, Farr S A, Verma S, Nonaka N, Nakamachi T, Ohtaki H, Niehoff M L, Edwards J C, Shioda S, Morley J E, Banks W A. Isolation of peptide transport system-6 from brain endothelial cells: therapeutic effects with antisense inhibition in Alzheimer and stroke models. J Cereb Blood Flow Metab. 2009 February; 29(2):411-22.

Falsini B, Marangoni D, Salgarello T, Stifano G, Montrone L, Campagna F, Aliberti S, Balestrazzi E, Colotto A. Structure-function relationship in ocular hypertension and glaucoma: interindividual and interocular analysis by OCT and pattern ERG. Graefes Arch Clin Exp Ophthalmol. 2008 August; 246(8):1153-62.

Hannibal J, Fahrenkrug J. Target areas innervated by PACAP-immunoreactive retinal ganglion cells. Cell Tissue Res. 2004 April; 316(1): 99-113.

Hannibal J, Brabet P, Fahrenkrug J. Mice lacking the PACAP type I receptor have impaired photic entrainment and negative masking. Am J Physiol Regul Integr Comp Physiol. 2008 December; 295(6):R2050-8.

John S W, Smith R S, Savinova O V, Hawes N L, Chang B, Turnbull D, Davisson M, Roderick T H, Heckenlively J R. Essential iris atrophy, pigment dispersion, and glaucoma in DBA/2J mice. Invest Ophthalmol Vis Sci. 1998 May; 39(6):951-62.

Kawaguchi C, Isojima Y, Shintani N, Hatanaka M, Guo X, Okumura N, Nagai K, Hashimoto H, Baba A. PACAP-deficient mice exhibit light parameter-dependent abnormalities on nonvisual photoreception and early activity onset. PLoS One. 2010 Feb. 18; 5(2):e9286

Kubrusly R C, da Cunha M C, Reis R A, Soares H, Ventura A L, Kurtenbach E, de Mello M C, de Mello F G. Expression of functional receptors and transmitter enzymes in cultured Muller cells. Brain Res. 2005 Mar. 21; 1038(2):141-9.

Kiss P, Tamás A, Lubics A, Lengvári I, Szalai M, Hauser D, Horvath Z S, Racz B, Gabriel R, Babai N, Toth G, Reglódi D. Effects of systemic PACAP treatment in monosodium glutamate-induced behavioral changes and retinal degeneration. Ann N Y Acad Sci. 2006 July; 1070:365-70.

Lee F S, Rajagopal R, Kim A H, Chang P C, Chao M V. Activation of Trk neurotrophin receptor signaling by pituitary adenylate cyclase-activating polypeptides. J Biol Chem. 2002 Mar. 15; 277(11): 9096-102.

Maffei L, Fiorentini A. Electroretinographic responses to alternating gratings in the cat. Exp Brain Res. 1982; 48(3):327-34.

Maffei L, Fiorentini A, Bisti S, Holländer H. Pattern ERG in the monkey after section of the optic nerve. Exp Brain Res. 1985; 59(2):423-5.

Nonaka N, Banks W A, Mizushima H, Shioda S, Morley J E. Regional differences in PACAP transport across the blood-brain barrier in mice: a possible influence of strain, amyloid beta protein, and age. Peptides. 2002 December; 23(12):2197-202.

Ozawa Y, Nakao K, Kurihara T, Shimazaki T, Shimmura S, Ishida S, Yoshimura A, Tsubota K, Okano H. Roles of STAT3/SOCS3 pathway in regulating the visual function and ubiquitin-proteasome-dependent degradation of rhodopsin during retinal inflammation. J Biol Chem. 2008 Sep. 5; 283 (36):24561-70.

Parisi V, Miglior S, Manni G, Centofanti M, Bucci M G. Clinical ability of pattern electroretinograms and visual evoked potentials in detecting visual dysfunction in ocular hypertension and glaucoma. Ophthalmology. 2006 February; 113(2):216-28.

Quigley H A, Dunkelberger G R, Green W R. Retinal ganglion cell atrophy correlated with automated perimetry in human eyes with glaucoma. Am J Ophthalmol. 1989 May 15; 107(5):453-64.

Rajagopal R, Chen Z Y, Lee F S, Chao M V. Transactivation of Trk neurotrophin receptors by G-protein-coupled receptor ligands occurs on intracellular membranes. J Neurosci. 2004 Jul. 28; 24(30):6650-8.

Seki T, Shioda S, Ogino D, Nakai Y, Arimura A, Koide R. Distribution and ultrastructural localization of a receptor for pituitary adenylate cyclase activating polypeptide and its mRNA in the rat retina. Neurosci Lett. 1997 Dec. 5; 238(3):127-30.

Seki T, Shioda S, Izumi S, Arimura A, Koide R. Electron microscopic observation of pituitary adenylate cyclase-activating polypeptide (PACAP)-containing neurons in the rat retina. Peptides. 2000 January; 21(1):109-13.

Seki T, Itoh H, Nakamachi T, Shioda S. Suppression of ganglion cell death by PACAP following optic nerve transection in the rat. J Mol Neurosci. 2008 November; 36(1-3):57-60.

Silveira M S, Costa M R, Bozza M, Linden R. Pituitary adenylyl cyclase-activating polypeptide prevents induced cell death in retinal tissue through activation of cyclic AMP-dependent protein kinase. J Biol Chem. 2002 May 3; 277(18): 16075-80.

Ventura L M, Porciatti V. Pattern electroretinogram in glaucoma. Curr Opin Ophthalmol. 2006 April; 17(2):196-202.

1. Topical ophthalmic formulations containing a carrier and a PACAP (Pituitary Adenylate Cyclase Activating Polypeptide) for the treatment of glaucoma-related visual deficits.

2. Formulations as claimed in claim 1, wherein PACAP is PACAP 27 or PACAP 38.

3. Formulations as claimed in claim 1, wherein the visual deficits are related to an early stage of glaucoma, before the onset of ganglion cell death.

4. Formulations as claimed in claim 1, in the form of solutions, suspensions, gels or ointments containing PACAP in concentrations ranging from 0.2 to 50 μg/μl.

5. Formulations as claimed in claim 1, wherein the carrier is a saline solution optionally containing viscosity-increasing agents selected from carboxymethylcellulose, Carbopol, hydroxypropylcellulose, polysaccharides, glycosaminoglycans and mixtures and derivatives thereof.

6. Formulations as claimed in claim 5 wherein the viscosity-increasing agent is tamarind seed polysaccharide (TSP).

7. Formulations as claimed in claim 6 wherein TSP is present in concentrations from 0.05 to 2% (weight/volume-w/v).

8. Formulations as claimed in claim 5 wherein the viscosity-increasing agent is sodium carboxymethylcellulose in percentages ranging from 0.01 to 2% (w/v).

9. Formulations as claimed in claim 5 wherein the viscosity-increasing agent is hyaluronic acid in concentrations from 0.05% to 0.8% (w/v), optionally in admixture with TSP.

10. Method of treating neurodegenerative diseases of the retina, optical nerve and lateral geniculate body in order to restore normal vision at an early stage of the diseases in patients in need thereof with a medicament in the form of eyedrops, said medicament comprising PACAP, said method comprising
administering to said patients in need thereof an effective amount of said medicament and
treating said patients in need thereof.
